(19) United States
(12) Patent Application Publication
(10) Pub. No.: US 2025/0290148 A1
(43) Pub. Date: Sep. 18, 2025

(54) DIFFERENTIAL ALTERNATIVE SPLICING IN RELAPSED AND REFRACTORY DIFFUSE LARGE-B CELL LYMPHOMA PATIENTS RECEIVING CAR-T THERAPY

(71) Applicant: H. LEE MOFFITT CANCER CENTER AND RESEARCH INSTITUTE INC., Tampa, FL (US)

(72) Inventors: Frederick L. Locke, Lutz, FL (US); Timothy Robinson, Madison, CT (US); Michael Jain, Tampa, FL (US); Jerald Noble, Dunnellon, FL (US); Thushara Madanayake, Woodrbidge, CT (US)

(21) Appl. No.: 18/860,399

(22) PCT Filed: Apr. 27, 2023
(86) PCT No.: PCT/US2023/066297

§ 371 Date: Oct. 25, 2024

Related U.S. Application Data
(60) Provisional application No. 63/363,669, filed on Apr. 27, 2022.

(51) Int. Cl. C12Q 1/6886; C12N 15/113

(52) U.S. Cl. CPC C12Q 1/6886; C12N 15/113; C12N 2310/11; C12N 2320/33; C12Q 2600/106; C12Q 2600/156

(57) ABSTRACT

Disclosed herein is a method for preventing or reversing CAR-T cell resistance and/or radioresistance in a relapsed and refractory diffuse large B-cell lymphoma (R/R DLBCL) of a subject, that involves assaying a sample from the subject for mRNA sequences of genes with roles in DNA damage, apoptosis, immune activation, and/or c-MYC signaling; detecting aberrant splicing in one or more of the mRNA sequences; and administering to the subject an antisense oligonucleotide (ASO) that prevents the aberrant splicing.

### CROSS-REFERENCE TO RELATED APPLICATIONS

This application claims benefit of U.S. Provisional Application No. 63/363,669, filed Apr. 27, 2022, which is hereby incorporated herein by reference in its entirety.

### SEQUENCE LISTING

This application contains a sequence listing filed in ST.26 format entitled “320803_2650_Sequence_Listing” created on Apr. 26, 2023. The content of the sequence listing is incorporated herein in its entirety.

### BACKGROUND

Chimeric antigen receptor (CAR) T cell therapy has recently emerged as an effective treatment for patients with relapsed and refractory (R/R) diffuse large B-cell lymphoma (DLBCL). However, the majority of patients ultimately progress and mechanisms of resistance to CAR T therapy remain poorly understood.

### SUMMARY

There are underlying molecular mechanisms of resistance to both radiation and CAR T-cell therapy in a substantial proportion of patients with Relapsed and/or Refractory Diffuse Large B-Cell Lymphoma (R/R DLBCL). As disclosed herein, differential native splicing (AS) events, specifically intron retention events within genes mediating apoptosis, serve as markers for response to radiation and CAR-T therapy in relapsed and refractory DLBCL patients. Specifically, aberrant splicing in genes with roles in DNA damage, apoptosis, immune activation, and c-MYC signaling are primary drivers of both CAR T and radiation resistance in patients with R/R DLBCL.

Therefore, disclosed herein is a method for preventing or reversing CAR-T cell resistance and/or radioresistance in a relapsed and refractory diffuse large B-cell lymphoma (R/R DLBCL) of a subject, that involves assaying a sample from the subject for mRNA sequences of genes with roles in DNA damage, apoptosis, immune activation, and/or c-MYC signaling; detecting aberrant splicing in one or more of the mRNA sequences; and administering to the subject an antisense oligonucleotide (ASO) that prevents the aberrant splicing.

For example, in some embodiments, the gene is an FBXW7 gene, and the aberrant splicing involves exon 2 retention, and the ASO promotes exon 2 skipping. Therefore, in some embodiments the ASO comprises the nucleic acid sequence

(SEQ ID NO: 1)
GGCCACTCACACTTTTAGAAAAGAG.

In some embodiments, the gene is CD19, the aberrant splicing involves intron 2 retention, and the ASO promotes intron 2 skipping. Therefore, in some embodiments the ASO comprises the nucleic acid sequence AACAGCTCCCCTGGGAAGAGACCCA (SEQ ID NO:2). In some embodiments, the gene is CD19, the aberrant splicing comprises intron 6 retention, and the ASO promotes intron 6 skipping.

In some embodiments, the gene is ATG16L1, the aberrant splicing involves intron 13 skipping, and the ASO promotes intron 13 retention. Therefore, in some embodiments the ASO comprises the nucleic acid sequence G ACTGAATTTCCTCACAGACTTTGC (SEQ ID NO:3).

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a summary of differential AS events. Y-axis is the −log(10) FDR and γ-axis is the change in PSI. Upregulated events are colored blue, downregulated events are coded red, and non-significant events are colored green. FIG. 1B is a summary of differential AS event types. FIGS. 1C and 1D show reads supporting the inclusion or skipping of the retained intron in CASP2 (FIG. 1C) or HMGB1 (FIG. 1D) in DR and NDR groups and the PSI of the retained intron in DR and NDR groups. FIG. 1D is the same as FIG. 1C but for the retained intron in HMGB1. FIG. 1E contains survival plots stratified by textiles of IR PSI in in CASP2 (FIG. 1E) or HMGB1 (FIG. 1F) indicating the progress free survival (left) and overall survival (right) following CAR-T therapy.

FIG. 2 shows Non-Hodgkins Lymphoma cell lines are characterized by one of the highest “levels of aberrant mRNA splicing as indicated by NHL-specific neojunctions.

FIG. 3 shows unsupervised clustering of ES events.

FIGS. 4A and 4B show custom ASOs that result in skipping of FBXW7 exon 2 result in increased sensitivity to both radiation (FIG. 4A) and CAR T-cell (FIG. 4B) killing. For the xCELLigence assay, OCI-Ly3 cells were plated at 0 hours. At 24 hours, ASOs were added. At 48 hours, CAR T-cells were added. Cell survival was assessed by resistance corresponding to the number of remaining cells attached to the plate.

FIG. 5A shows IGV mapping of reads confirms low vs. high intron retention as predicted by RMATs. FIG. 5B is a radar plot of CD19 intron retention (all 14 introns shown). FIG. 5C shows retention introns 2 or 6 were each associated with poorer PFS, and confirmed to occur in cell lines (CCLE) and two distinct clinical cohort.

FIGS. 6A to 6F show inclusion levels of CD19 introns 2 and 6 and the percentage of transcripts excluding CD19 introns 2 and 6 in DR and NDR patients. FIGS. 6A and 6B show PSI values of intron 2 and 6 inclusion, ΔPSI between DR and NDR patients, and associated FDR value calculated by rMATS. FIG. 6C shows the percentage of transcripts excluding CD19 introns 2 and 6 calculated using PSI values generated by rMATS in DR and NDR patients and the ΔPSI and p-value comparing DR and NDR patients. FIGS. 6D to 6E shows PSI values of intron 2 and 6 inclusion, ΔPSI between DR and NDR patients, and associated Wilcoxon p-value calculated by our pipeline. FIG. 6F shows the percentage of transcripts excluding CD19 introns 2 and 6 calculated using PSI values generated by our pipeline in DR and NDR patients and the ΔPSI and p-value comparing DR and NDR patients.

FIG. 7A shows predictive performance of logistical regression model using the percentage of normal CD19 transcripts. Y-axis denotes the probability of DR and X-axis denotes the percentage of normal CD19 transcripts in a sample. FIG. 7B shows) ROC curve for the same model in FIG. 7A. FIG. 7C shows progress free survival for patients above and below the median expression level of percent normal CD19 transcripts in the population. FIG. 7D is a summary survival statistics for the same patients in FIG. 7C.

FIGS. 8A to 8L show the relation between the gene expression of six RNA binding proteins with CD19 intron 6 retention. FIGS. 8A to 8F show relation between the genes and CD19 intron 6 retention in the CAR-T treatment cohort. FIGS. 8G to 8L show relation between the genes and CD19 intron 6 retention in the NCICCR-DLBCL cohort.

FIG. 9 shows a sashimi plot depicting the median number of reads supporting the splicing out of CD19 intron 2 (upper arc) and the median read coverage CD19 intron 2 in DR and NDR patients.

FIG. 10 shows a sashimi plot depicting the median number of reads supporting the splicing out of CD19 intron 6 (upper arc) and the median read coverage CD19 intron 6 in DR and NDR patients.

FIGS. 11A to 11D show the correlation of CD19 gene expression to CD19 intron 6 retention levels. FIG. 11A shows a CAR-T patient cohort. FIG. 11B shows a NCICCR-DLBCL cohort. FIG. 11C shows a TCGA-DLBCL cohort. FIG. 11D shows CCLE-DLBCL cell lines.

FIGS. 12A to 12D show the correlation of CD19 gene expression to CD19 intron 6 retention levels. FIG. 12A shows a CAR-T patient cohort. FIG. 12B shows a NCICCR-DLBCL cohort. FIG. 12C shows a TCGA-DLBCL cohort. FIG. 12D shows CCLE-DLBCL cell lines.

FIG. 13 shows CD19 intron 6 retention levels in DR and NDR patients in the CAR-T cohort, CCLE-DLBCL cell lines, NCICCR-DLBCL cohort, and TCGA-DLBCL cohort.

FIG. 14 Shows CD19 intron 2 retention levels in DR and NDR patients in the CAR-T cohort, CCLE-DLBCL cell lines, NCICCR-DLBCL cohort, and TCGA-DLBCL cohort.

FIG. 15 shows UCSC genome browser tracks for CD19. Dotted rectangular box denotes the location of intron 6.

FIG. 16 shows The relation between the gene expression of six RNA binding proteins with CD19 intron 2 retention. (A-F) Relation between the genes and CD19 intron 2 retention in the CAR-T treatment cohort. (G-L) Relation between the genes and CD19 intron 2 retention in the NCICCR-DLBCL cohort.

FIG. 17 shows number of IR events detected in DR & NDR patients. Detection=PSI>0.

FIG. 18 shows number of IR events detected in DR & NDR patients. Detection=PSI>0.1.

FIG. 19 shows correlation of the number of intron retention events detected in a patient and CD19 intron 6 PSI.

FIG. 20 shows the correlation of significantly differentially spliced IR events using scaled PSI values. Dotted lines highlight IR in CD19.

FIG. 21 shows the correlation of significantly differentially spliced IR events using non-scaled PSI values. Dotted lines highlight IR in CD19.

FIGS. 22A to 22H shows the inclusion levels of CD19 introns 2 and 6 and the percentage of transcripts excluding CD19 introns 2 and 6 in DR and NDR patients. FIGS. 12A to 12B show PSI values of intron 2 using rMATS (FIG. 12A) or a custom IR quantification pipeline (FIG. 12B). FIG. 12C is a 9 Sashimi plot depicting the median number of reads supporting the splicing out of CD19 intron 2 (upper arc) and the median read coverage CD19 intron 2 in DR and NDR patients. FIGS. 12D to 12E show PSI values of intron 6 using rMATS (FIG. 12D) and the custom IR quantification pipeline (FIG. 12E). FIG. 12F is a Sashimi plot depicting the median number of reads supporting the splicing out of CD19 intron 2 (upper arc) and the median read coverage CD19 intron 2 in DR and NDR patients. FIGS. 12G to 12H show percentage of isoforms not expression CD19 intron 2 and intron 6 using rMATS PSI values (FIG. 12G) and the custom IR quantification pipeline (FIG. 12H).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

As used herein, the term “complementary”, refers to the capacity for precise pairing between two nucleotides. An antisense oligonucleotide (ASO) may be at least 80% complementary to (optionally one of at least 85%, 90%, 91%, 92%, 93%, 94%, 95%, 96%, 97%, 98%, 99% or 100% complementary to) the consecutive nucleotides of a human progranulin gene. In some embodiments, the ASO may contain 1, 2, or 3 base mismatches compared to the portion of the consecutive nucleotides of a GRN-associated region. In some embodiments the single stranded oligonucleotide may have up to 3 mismatches over 15 bases, or up to 2 mismatches over 10 bases. It is understood in the art that a complementary nucleotide sequence need not be 100% complementary to that of its target to be specifically hybridizable. In some embodiments, a complementary nucleic acid sequence for purposes of the present disclosure is specifically hybridizable when binding of the sequence to the target molecule (e.g., pre-mRNA) interferes with the normal function of the target (e.g., pre-mRNA) to cause a loss of activity and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

As used herein, the term “nucleotide” refers to an organic molecule that serves as the monomer unit for forming the nucleic acid polymers deoxyribonucleic acid (DNA) and ribonucleic acid (RNA). Nucleotides are the building blocks of nucleic acids and are composed of three subunit molecules: a nitrogenous base, a five-carbon sugar, and at least one phosphate group. Nucleotides can be modified. The preparation of modified nucleic acids, backbones, and nucleobases described above are well known in the art. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Modified nucleotides can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2—NH—CH2—, —CH2—N(CH3)—O—CH2—[known as a methylene (methylimino) or MMI backbone], —CH2—O—N(CH3)—CH2—, —CH2—N(CH3)—N(CH3)—CH2— and —N(CH3)—CH2—CH2—[wherein the native phosphodiester backbone is represented as —O—P—O—CH2—].

As used herein, an “antisense oligonucleotide (ASO)” refers to a synthesized nucleic acid sequence that is complementary to a target DNA or mRNA sequence. Antisense oligonucleotides are typically designed to increase expression of a DNA or RNA target by binding to the target and modulation the expression or activity at the level of transcription, translation, or splicing. Antisense oligonucleotides are generally designed to hybridize under cellular conditions to a gene, e.g., the progranulin gene, or to its transcript. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits progranulin may comprise at least 5, at least 10, at least 15, at least 20, at least 25, at least 30, or more bases complementary to a portion of the coding sequence of the human progranulin gene (e.g., NCBI Gene ID: 2896), respectively.

As used herein, an “exon” refers to any part of a gene that will encode a part of the final mature RNA produced by that gene after introns have been removed by RNA splicing. The term exon refers to both the DNA sequence within a gene and to the corresponding sequence in RNA transcripts.

As used herein, an “intron” refers to any nucleotide sequence within a gene that is removed by RNA splicing during maturation of the final RNA product. The term intron refers to both the DNA sequence within a gene and the corresponding sequence in RNA transcripts. Group I and group II introns are found in genes encoding proteins (messenger RNA), transfer RNA and ribosomal RNA in a very wide range of living organisms. Following transcription into RNA, group I and group II introns also make extensive internal interactions that allow them to fold into a specific, complex three-dimensional architecture. These complex architectures allow some group I and group II introns to be self-splicing, that is, the intron-containing RNA molecule can rearrange its own covalent structure so as to precisely remove the intron and link the exons together in the correct order.

As used herein, the term “alternative splicing” refers to a regulated process during gene expression that results in a single gene coding for multiple proteins. In this process, particular exons of a gene may be included within or excluded from the final, processed messenger RNA (mRNA) produced from that gene.

As used herein, the term “exon skipping” refers to an exon that may be spliced out of the primary transcript or retained.

As used herein, the term “Intron retention” refers to a sequence may be spliced out as an intron or simply retained. This is distinguished from exon skipping because the retained sequence is not flanked by introns.

As used herein, the term “gapmer” refers to a chimeric antisense oligonucleotide that contains a central block of deoxynucleotide monomers sufficiently long to induce RNase H cleavage.

The term “therapeutically effective amount” refers to an amount of the ASOs described herein, using the methods as disclosed herein, that is sufficient to provide a particular effect when administered to a typical subject. An effective amount as used herein would also include an amount sufficient to delay the development of a symptom of the disease, alter the course of a symptom of a disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

Disclosed herein is a method for preventing or reversing CAR-T cell resistance and/or radioresistance in a relapsed and refractory diffuse large B-cell lymphoma (R/R DLBCL) of a subject. In some embodiments, the method involves assaying a sample from the subject for mRNA sequences of genes with roles in DNA damage, apoptosis, immune activation, and/or c-MYC signaling. In some embodiments, the method further involves detecting aberrant splicing in one or more of the mRNA sequences. In some embodiments, the method further involves administering to the subject an antisense oligonucleotide (ASO) that prevents the aberrant splicing.

### Genes with Roles in DNA Damage, Apoptosis, Immune Activation, and/or c-MYC Signaling

In some embodiments, any gene with a role in DNA damage, apoptosis, immune activation, and/or c-MYC signaling known in the art and can be used in the disclosed methods.

In some embodiments, the gene is selected from the group consisting of TBL2, CLK1, ZMIZ2, NCOA6, NKTR, ASDURF, NFYA, EXOC7, HTRA2, EWSR1, TLR10, SIKE1, IRF3, ALG13, PRANCR, SMYD5, RALGDS, EPC1, ZHX1-C8orf76, TLK2, ACP1, NDUFS1, GRK5, MYNN, VPS53, DNAJB2, HMGN1, ANGEL2, ANKRD36, KANSL3, PRKDC, TRAF5, LRRCC1, PRDM15, LRRCC1, GUSBP11, POLR2H, PTGES3L-AARSD1, TPST1, CRTC2, RNF121, ACP1, HMGN1, GAS5, ZNF107, TRMT2B, PDK1, SCAMP1, GTF2H1, PTAR1, MVK, GAS5, AMPD3, SUN1, GAS5, COMMD3-BMI1, TRMT2B, SERGEF, BRWD1, ZSCAN30, SUN1, ARHGAP26, ENOX2, POLK, NT5C3A, CERT1, HMGN1, DTWD1, KANSL2, KAT6B, ZNF337, CNOT6, RMC1, CEP95, CTDSP2, SLC9A8, STYXL1, ZNF107, WDR61, HAUS1, SNX14, PDSS1, WDSUB1, AD000671.2, SERGEF, HMGCL, TTN-AS1, ZNF655, CIRBP, DDX17, LST1, RFX5, CYP4V2, CRTC2, EWSR1, HTRA2, CDK10, CLK1, AC005192.1, HLA-F, TRA2A, ORMDL1, CD19, UQCRC1, PEX1, RIPK3, TRMT13, CREBZF, MGAT4B, TROAP, GLT8D1, HERPUD1, SUN2, TAMM41, SNHG7, NDUFA3, CARMIL2, HMGN1, PYM1, ARNT, CSNK1A1, MAST3, ZMIZ1, TLR10, BPTF, ARNT, BPTF, ACP1, ZMIZ1, TBL2, ZMIZ1, HMGN1, DMTF1, RBMX, UBE2D3, ZMIZ1, RBMX, PSMA3-AS1, MEF2A, XPNPEP3, COP1, BPTF, SYNRG, MTRR, BPTF, RHOH, JKAMP, TMPO, PPP2R3C, MTRR, SS18, PXK, STAU1, LRR1, PPP2R3C, CNTRL, RIPOR2, MECP2, PIP5K1C, KLHL24, AZIN1, BTBD10, CNTRL, PPP6R2, MYO9A, NABP1, BTBD10, ZNF107, STAMBP, HMGN1, SERGEF, ING2, SCAI, and RB1CC1

### Assaying for Aberrant Splicing

Various methods are well known within the art for determining levels of properly spliced and aberrantly spliced extracellular transcripts. These methods can include identification and/or isolation and/or purification of a transcript from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample can be isolated according to standard methods, for example using filtration, centrifugation, or other methods of purification to obtain a sample that contains extracellular transcripts but does not contain cells or cellular transcripts. The methods can include using chemical solutions nucleic acid-binding resins following the manufacturer's instructions.

The transcripts can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of different splice isoforms. multiple-exon-skipping detection assay (MESDA) can also be used (see Singh et al., 2012, PLoS One. 2012; 7(11):e49595).

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof, which detect various spliced isoforms. The probe sequences can be synthesized either enzymatically in vivo, enzymatically in vitro (e.g. by PCR), or non-enzymatically in vitro.

### Antisense Oligonucleotides

Exon-skipping antisense oligonucleotides (ASOs) that correct missplicing can be used, e.g., as described in Siva et al., Nucleic Acid Ther. 2014 Feb. 1; 24(1): 69-86; Scotti and Swanson, Nature Reviews Genetics 17:19-32 (2016). For example, bicyclic-locked nucleic acids (LNAs), ethylene-bridged nucleic acids (ENAs), 2′-O-methyl phosphorothioate AO (2OME-PSs), peptide nucleic acids (PNAs), or phosphorodiamidate morpholino oligomers (PMOs) have been described that correct missplicing in clinical trials and animal models; see, e.g., Brolin and Shiraishi, Artif DNA PNA XNA. 2011 January-March; 2(1): 6-15; Scotti and Swanson, Nature Reviews Genetics 17:19-32 (2016); Touznik et al., Expert Opin Biol Ther. 2014 June; 14(6):809-19. The ASOs can be delivered, e.g., parenterally in liposomal complexes, e.g., cationic lipoplexes, or using a viral vector, e.g., a lentivirus, adenovirus, or adeno-associated virus. See e.g., Jarver et al., Nucleic Acid Ther. 2014; 24(1):37-47; Aartsma-Rus et al., Hum Gene Ther. 2014; 25(10): 885-892, McNally and Wyatt, J Clin Invest. 2016 Apr. 1; 126(4):1236-8; Imbert et al., Genes 2017, 8(2), 51; doi:10.3390/genes8020051.

Exon skipping uses antisense oligonucleotides (ASOs) to alter transcript splicing; the present methods can be used to detect these transcripts with desired splicing. These treatments can include antisense oligonucleotide-targeted exon skipping to induce near normal, e.g., for dystrophin, e.g., as described in Aartsma-Rus, Methods Mol Biol. 2012; 867:97-116. Clinical trials of ASOs in DMD have been conducted, see, e.g., Koo and Wood, Hum Gene Ther. 2013 May; 24(5):479-88; Voit et al., Lancet Neurol. 2014; 13(10):987-996.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions, methods, and uses that exhibit large therapeutic indices are preferred. A therapeutically effective dose can be estimated initially from cell culture assays. Also, a dose can be formulated in animal models to achieve a circulating plasma concentration range that includes the IC50, which achieves a half-maximal inhibition of measured function or activity as determined in cell culture, or in an appropriate animal model. The effects of any particular dosage can be monitored by a suitable bioassay. The dosage can be determined by a physician and adjusted, as necessary, to suit observed effects of the treatment.

### Administration

The agents described herein can be administered to a subject in need thereof by any appropriate route which results in an effective treatment in the subject. Exemplary modes of administration of the ASOs for the modulation of progranulin expression or activity in the brain by the ASO and/or ASOs disclosed herein include oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular (including administration to skeletal, diaphragm and/or cardiac muscle), intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

In some embodiments, the pharmaceutical compositions can conveniently be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

Liquid dosage forms include solutions, suspensions and emulsions. Liquid form preparations may be administered by intravenous, intracerebral, intraperitoneal, parenteral or intramuscular injection or infusion. Sterile injectable formulations may comprise a sterile solution or suspension of the active agent in a non-toxic, pharmaceutically acceptable diluent or solvent. Suitable diluents and solvents include sterile water, Ringer's solution and isotonic sodium chloride solution, etc. Liquid dosage forms also include solutions or sprays for intranasal administration.

Aerosol preparations suitable for inhalation may include solutions and solids in powder form, which may be combined with a pharmaceutically acceptable carrier, such as an inert compressed gas. Long-term release, are used herein, means that the implant is constructed and arranged to delivery therapeutic levels of the active ingredient for at least 5 days, for at least 10 days, for at least 15 days, for at least 20 days, for at least 30 days, for at least 40 days, for at least 50 days or for at least 60 days. Long-term sustained release implants are well-known to those of ordinary skill in the art and include some of the release systems described above.

Administration of the ASOs can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye.

Pharmaceutical preparations may be conveniently prepared in unit dosage form, according to standard procedures of pharmaceutical formulation. The quantity of active compound per unit dose may be varied according to the nature of the active compound and the intended dosage regime.

Other delivery systems can include time-release, delayed release or sustained release delivery systems. Such systems can avoid repeated administrations of the therapeutic agents of the invention described herein, increasing convenience to the subject and the physician. Many types of release delivery systems are available and known to those of ordinary skill in the art. They include polymer based systems such as poly(lactide-glycolide), copolyoxalates, polycapro-lactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the therapeutic agent(s) of the invention are contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Advantageously, the time of administration can be coupled with other treatment methodologies. The above agents may also be used in combination in order to achieve the desired therapeutic effect. Certain combinations of agents may act co-operatively, additively or synergistically, when co-administered or when administered sequentially. The antisense treatment may be applied before, after, or in combination with other treatments.

In some embodiments, Cell-penetration peptides (CPPs) can be used as a transmembrane drug delivery agent for improved delivery of ASOs targeting a 5′ untranslated region of progranulin. CPPs are a class of small cationic peptides of at least 10, or at least 11, or at least 12, or at least 13, or at least 14, or at least 15, or at least 15, or at least 20, or at least 25, or at least 30 amino acids that can be used as transmembrane drug delivery agents through various forms of endocytosis for low-molecular weight compounds, including drugs, imaging agents, oligonucleotides, peptides and proteins. CPPs are also known as ‘protein transduction domains’. CPPs include but are not limited to the peptides Tat or penetratin.

In some embodiments, arginine-rich CPPs can be used for improved delivery of ASOs targeting progranulin to the brain, e.g. Pep-3, for in vivo delivery.

For example, in some embodiments, the gene is an FBXW7 gene, and the aberrant splicing involves exon 2 retention, and the ASO promotes exon 2 skipping. Therefore, in some embodiments the ASO comprises the nucleic acid sequence

(SEQ ID NO: 1)
GGCCACTCACACTTTTAGAAAAGAG.

In some embodiments, the gene is CD19, the aberrant splicing involves intron 2 retention, and the ASO promotes intron 2 skipping. Therefore, in some embodiments the ASO comprises the nucleic acid sequence AACAGCTCCCCTGGGAAGAGACCCA (SEQ ID NO:2). In some embodiments, the gene is CD19, the aberrant splicing comprises intron 6 retention, and the ASO promotes intron 6 skipping.

In some embodiments, the gene is ATG16L1, the aberrant splicing involves intron 13 skipping, and the ASO promotes intron 13 retention. Therefore, in some embodiments the ASO comprises the nucleic acid sequence G ACTGAATTTCCTCACAGACTTTGC (SEQ ID NO:3).

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Methods

A cohort of 32 R/R DLBCL were profiled with RNAseq prior to receipt of CAR T therapy. Differences in pre-treatment mRNA alternative splicing (AS) were compared between 17 patients who exhibited a durable response (DR) vs. 15 with non-durable response (NDR) using rMATS and allowing for discovery of novel AS events.

### Results

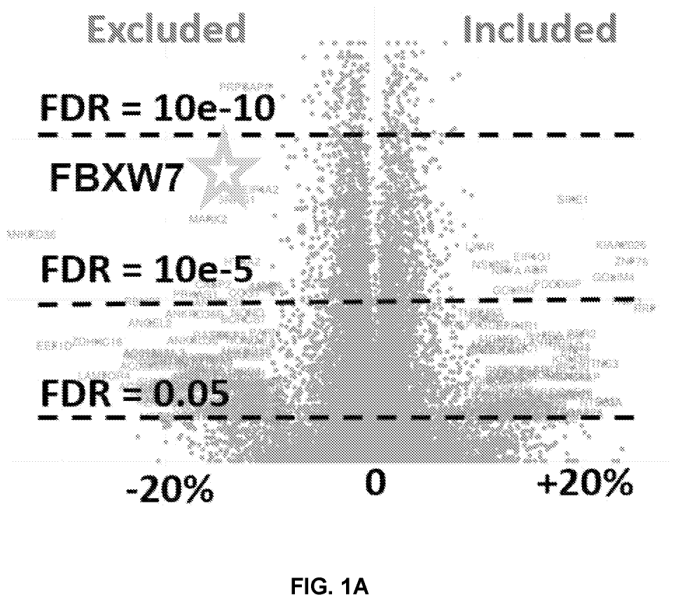

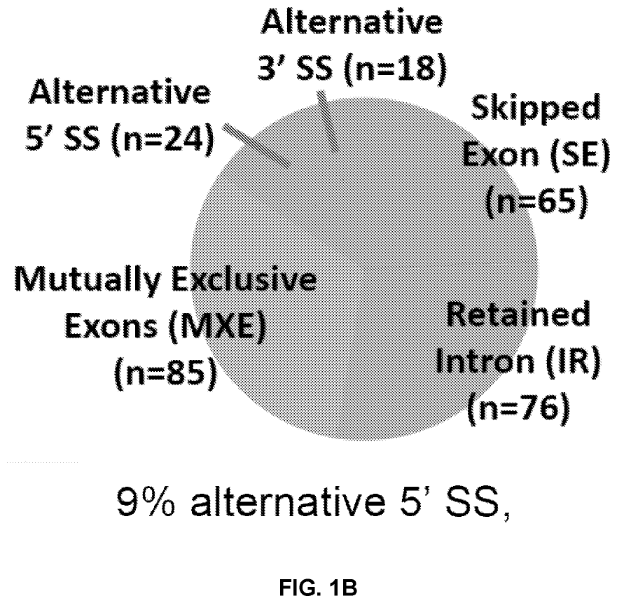

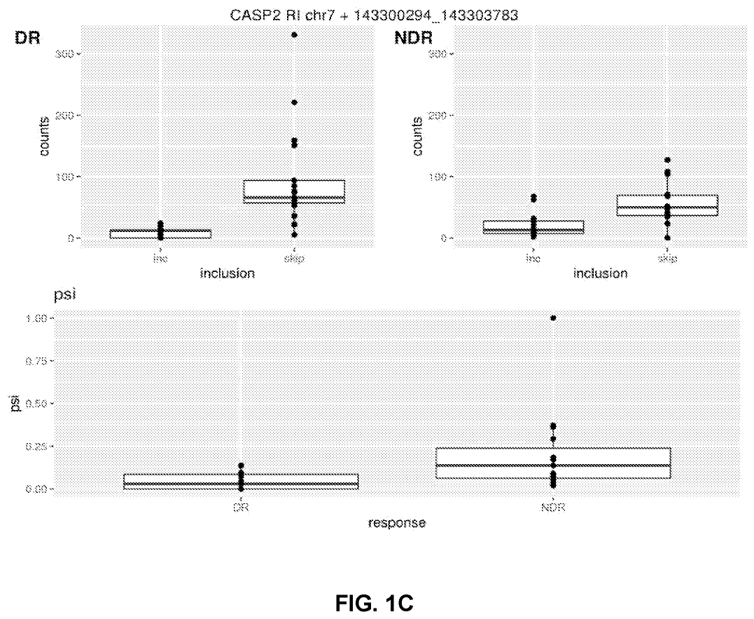

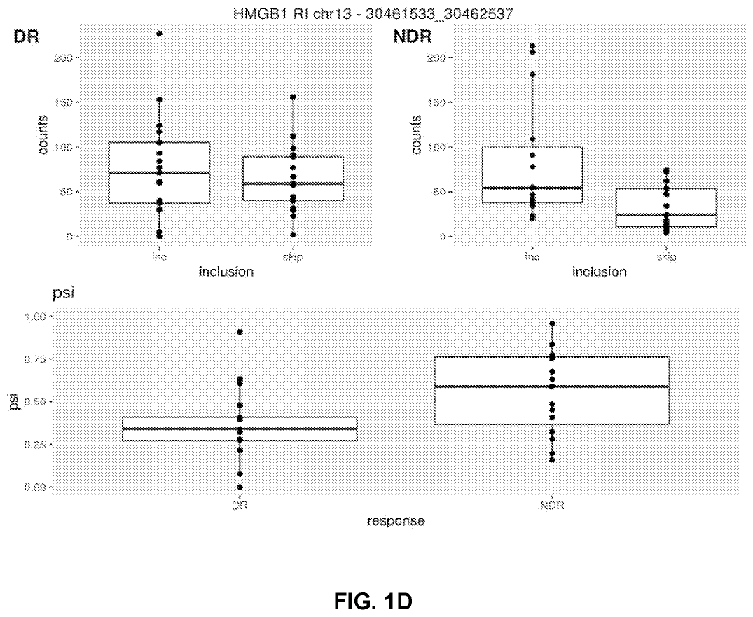

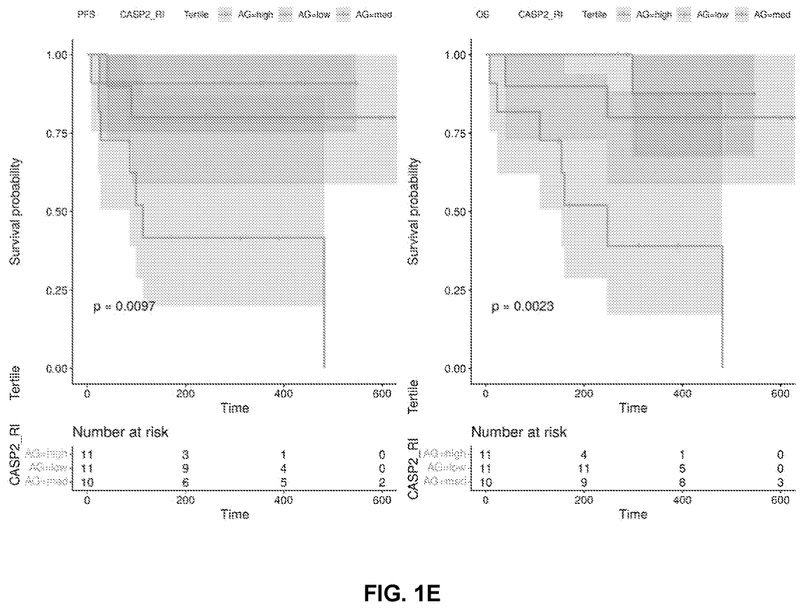

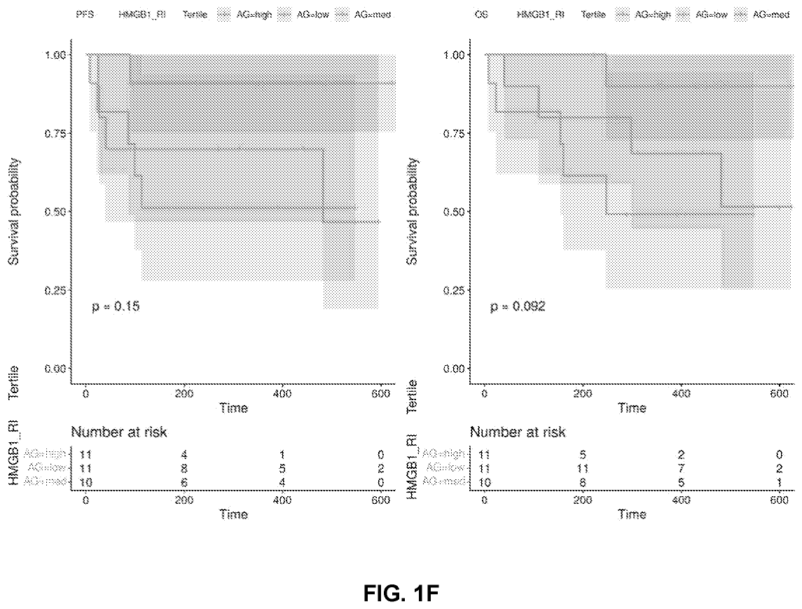

268 dAS events were identified at an FDR adjusted p-value <0.05 and a change in percent spliced in (PSI) of 10% occurring in 209 genes (FIG. 1A). Among these AS events, 28.4% were intron retentions (IR), 31.7% were mutually exclusive exons, 24.3% were exon skips, 9% were alternative 5′ splice sites, 6.7% were alternative 3′ splice sites (FIG. 1B). A GO term enrichment for the parent genes of these AS events yielded an overrepresentation of DNA damage response (fold enrichment: 8.49; FDR: 0.009) and negative regulation of G1/S transition (fold enrichment: 6.27; FDR: 0.036). To further refine which biological processes were enriched among AS types, the same analyses was performed using the parent genes of each AS event type. IR events were significantly enriched for pathways in toll-like receptor 4 signaling (enrichment: 36.01; FDR: 0.043) and apoptosis (enrichment: 17.9; FDR: 0.04). The most significant hits among these two biological processes were IR events in HMGB1 and CASP2, respectively. The IR event in CASP2 was 15.5% more retained in NDR patients (FDR: 3.3e-6) (FIG. 1C) and the event in HMGB1 was 19.6% more highly retained in NDR patients (FDR: 0.005) (FIG. 1D). High levels of CASP2 IR were associated with poor survival outcomes (FIG. 1E) while the effect of HMGB1 IR on survival outcomes was less clear (FIG. 1F). Cross referencing of hits with a functional screen of CAR-T resistance confirmed 9 overlapping targets and shared an enrichment for apoptosis-related pathways.

### Conclusions

These results indicate that differential AS events, specifically intron retention events within genes mediating apoptosis, may serve as markers for response to CAR-T therapy in relapsed and refractory DLBCL patients and are being pursued in ongoing studies.

TABLE 1

| Event Type | Number of Hits | Number of Genes |
|---|---|---|
| IR | 77 | 75 |
| ES | 66 | 47 |
| MXE | 86 | 73 |
| A5 | 25 | 19 |
| A3 | 19 | 17 |

### Example 2

### Results

### DLBCL is Enriched for Aberrant Splicing

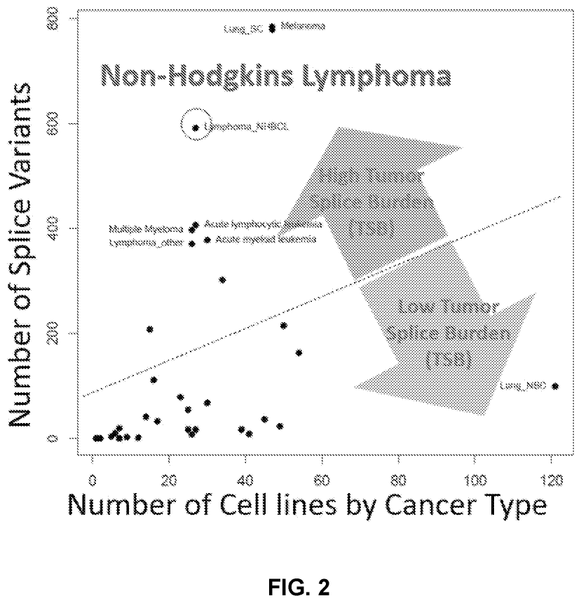

Aberrant mRNA splicing is a widespread phenomenon in cancer (FIG. 2). a preliminary pipeline was constructed to identify aberrant mRNA splicing from RNA-seq data and analyzed RNA-seq data from 1,457 cell lines using the CCLE (Cancer Cell Line Encyclopedia), which is a publicly available resource hosted by the Broad Institute. RNA-seq Junctions were removed if they had less than 20 reads support, less than 10% percent use, or overlapped normal tissue (i.e. GTEx) junctions. Several thousand cancer-specific aberrant splicing events distributed across the various disease sites were observed. Different disease sites had different numbers of data samples and so we have noted the number of cell lines for each CCLE cancer type as well. Interestingly, some cancer types had higher numbers of cancer-specific aberrant splicing events or had a higher “tumor splice burden” than other cancer types. In particular, a large number of cancer-specific events were observed within small cell lung cancer (778 events), melanoma (784 events), and several hematologic malignancies including AML (378), Non-Hodgkins Lymphoma (592), ALL (407), multiple myeloma (397), and other lymphomas (371). The number of neojunctions were then normalized by the number of cell lines analyzed in order to measure a “tumor splice burden” for each cancer type represented with the CCLE. Cell lines from hematologic malignancies (AML, ALL, NHBCL, MM, and other lymphoma) represented 5 of the top 7 cancer types, each of which had over 300 neojunctions that were unique to that specific cancer. 592 neojunctions were detected in a total of 27 Non-Hodgkin's lymphoma cell lines, suggesting one of the highest levels of tumor splice burden across cancer types (FIG. 2).

Zhang et al. conducted an investigation of mRNA splicing in the TCGA DLBCL cohort using SpliceSeq with respect to the impact of splicing on survival and reported that SF1, and several HNRNP genes (C, D, H3) were implicated in Splicing events that were associated with overall survival, suggesting the presence of aberrant splicing in human DLBCLs (Ryan M C, et al. Bioinformatics 2012 28(18):2385-7). None of these patients received CAR T therapy, no clinical or treatment data was included, and many different approaches were used to generate correlative signatures with no functional or secondary validation conducted. In their reported analysis they were not able to detect any clinically significant intron retention (IR) or exon skipping events(ES).

Numerous Aberrant mRNA Splicing Events are Significantly Associated with a Lack of Durable Clinical Response to CD19-Directed CAR T-Cell Therapy in R/R DLBCL

Differential AS analysis was performed in a cohort of 32 patients with relapsed and refractory DLBCL who received CAR-T therapy. Seventeen patients exhibited a durable response following treatment (DR) and 15 did not exhibit a durable response (NDR). Differential AS analyses between DR and NDR samples was performed using rMATS (Shen et al. 2014) allowing the discovery of novel AS events to identify statistically significant changes in alternative splicing between DR and NDR samples. Percent spliced in (PSI), or the percent of reads spanning a junction as a function of all reads covering both junctions, was calculated for IR and exon skipping events that were identified as differentially spliced by rMATS (Shen et al. 2014). 268 differential AS events at an FDR adjusted p-value <0.05 and a change in percent spliced in (PSI) of 10% occurring in 209 genes were identified (FIG. 1A volcano plot). Among these AS events, 28.4% were IRs, 31.7% were mutually exclusive exons, 24.3% exon skipping, 6.7% alternative 3′ splice site (FIG. 1B).

Unsupervised Clustering of Splicing Events Indicates Aberrant mRNA Splicing Profiles are Associated with Durable Responses to CAR T Therapy

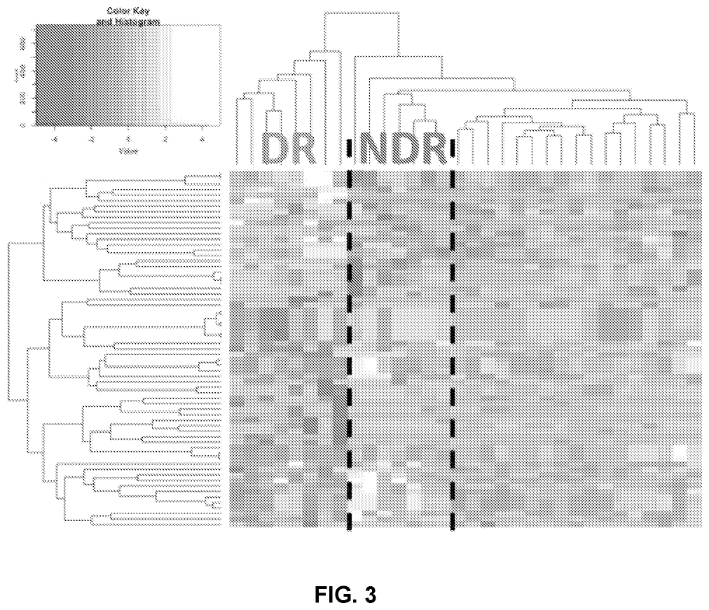

In order to examine whether there was any inherent relationship between splicing events and outcomes, unsupervised hierarchical clustering of both IR & ES events was, which both resulted similar clustering of patients, suggesting a common underlying biology regulating aberrant splicing within a subset of patients. Clustering by ES events, with no supervision, revealed distinct clusters of patients by response status including 8 patients with durable response (FIG. 3, Left group), a second cluster of 7 patients without any durable responses (2nd from left group), and remaining patients who further tended to cluster by response rates by ES event prevalence (right group).

Identified Splicing Events Associated with CAR T Resistance are Enriched in Genes with Roles in DNA Damage Repair, Apoptosis, Immune Activation, and c-MYC Signaling

A gene ontology term enrichment for the parent genes of these AS events yielded an overrepresentation of the biological processes “DNA damage response, signal transduction by a p53 class mediator” (fold enrichment: 8.49; FDR: 0.009) and “negative regulation of G1/S transition of mitotic cell cycle” (fold enrichment: 6.27; FDR: 0.036). To further refine which biological processes were enriched among AS types, the same analyses was performed using the parent genes of each AS event type. Intron retained (IR) genes showed significant enrichment and the top enriched biological processes were “regulation of toll-like receptor 4 signaling pathway” (enrichment: 36.0; FDR: 0.043) and “execution phase of apoptosis” (enrichment: 17.9; FDR: 0.04). The parent genes of the differential AS events were cross referenced to an in vitro CRIPSR genome-wide KO study by Singh et al. (2020) 11 investigating resistance to CAR-T therapy in patients with ALL. Nine of the splicing events identified in the study were also identified in this functional screen of CAR T resistance, thus confirming functional roles of genes identified in CAR T resistance. Based on these results, the central hypothesis was that aberrant mRNA splicing events in genes with roles in DNA damage, apoptosis, immune activation, and c-MYC signaling are primary drivers of CAR T and radiation resistance in patients with R/R DLBCL.

FBXW7 exon 2 skipping can be induced by antisense oligonucleotides (ASOs) and results in re-sensitization to both CAR T-cell killing and radiation. One of the top splicing events correlated with CAR T-cell resistance based on statistical significance and relevant literature was FBXW7 exon 2 inclusion. FBXW7 is a component of the E3 ubiquitin ligase Skp1-Cullin1-F-box (SCF) and the identified aberrantly spliced exon 2 is located within the domain that determines which substrates are targeted for degradation (Yeh C H, et al. Molecular cancer. 2018 17(1):115). The SCF ubiquitin ligase complex engages in proteolytic degradation of ubiquinated proteins to regulate cell cycle, cell growth, and apoptosis. FBXW7 is a known tumor suppressor in multiple cancers due to its role in targeting several oncogenes for degradation, including c-Myc, Notch, cyclin E, c-JUN, NF-kB, and KLF568,69. FBXW7 is located on a gene region, chromosome 4q32, that is deleted in 30% of human cancers, and aberrations in FBXW7 have been identified in brain cancer, breast cancer, colorectal cancer and leukemias. TALL can be induced by a mutation in FBXW7 alone, independent of other tumor-promoting mutations. FBXW7 was shown to attenuate innate immune response via HMGB1 degradation.

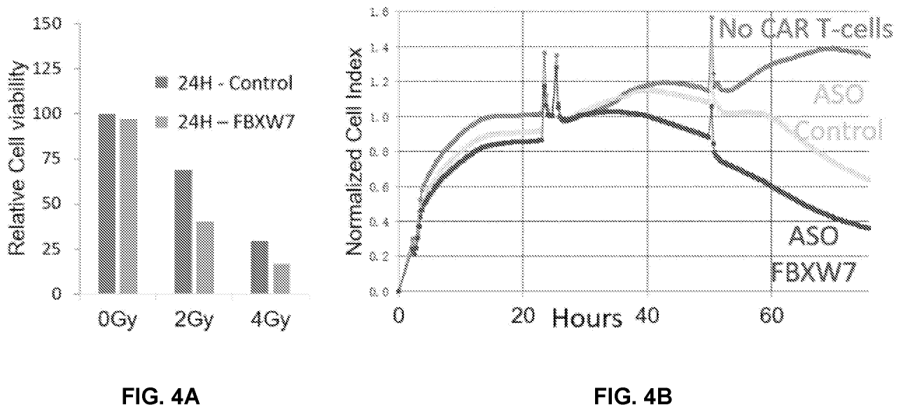

The occurrence of the FBXW7 exon 2 skipping event was confirmed in silico in two external DLBCL patient cohorts (TCGA 70 & NCICCR-DLBCL 71) and CCLE lymphoma cell lines with high levels of exon 2 inclusion (Toledo, Jeko1, EJ-1, and OCI-Ly3). The identification of lymphoma cell lines with this aberrant splicing event allowed for study of the impact of FBXW7 exon 2 skipping. Custom 25 bp custom ASOs chemically formulated for in vivo use (no transfection agent required) were used to target the 5′ splice site of FBXW7 and RT-PCR across exons 1-3 was used to confirm the ability to induce near-complete exon 2 skipping. Custom ASO-induced skipping of exon 2 in OCI-Ly3 cells was able to restore sensitivity to ionizing radiation and sensitivity to in vitro CAR T-cell killing (FIG. 4) using our xCELLigence assay (see additional experimental details). This confirms a functional role of exon 2 inclusion/exclusion in mediating BOTH radiation and CAR T-cell sensitivity.

CD19 Intron 2 and 6 Retention are Associated with Clinical CAR T-Cell Resistance

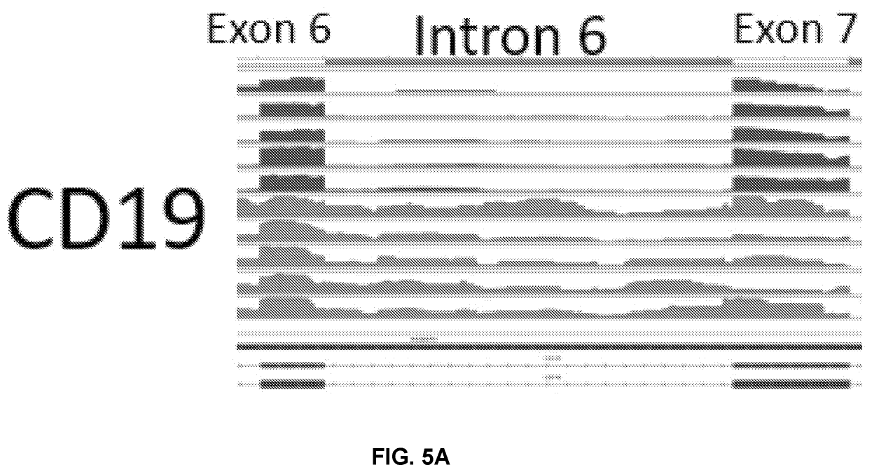

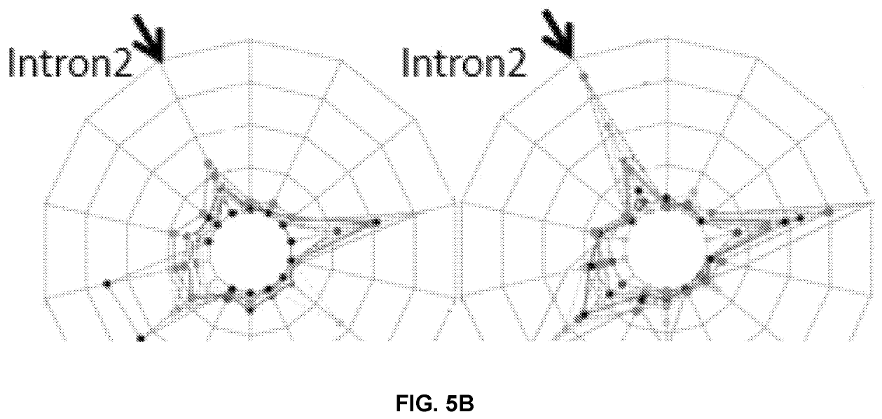

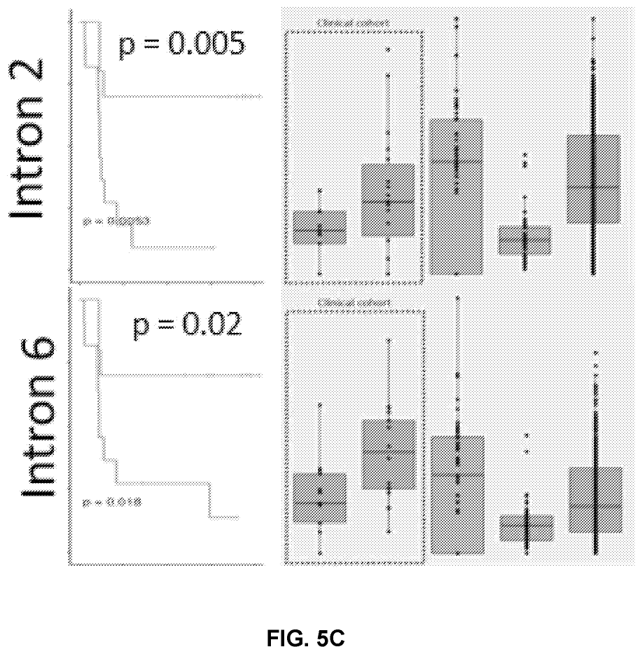

Resistance to CD19 CAR T therapy in B-cell leukemia occurs in part via aberrant CD19 splicing, in which leukemic cells have been observed to skip CD19 exon 2, thereby splicing out immunogenic epitopes without losing function of the entire oncogene (Song M K, et al. International journal of molecular sciences. 2019 20(20); Xu X, et al. Frontiers in immunology. 2019 10:2664). However, CD19 exon 2 skipping has not been correlated with CAR T efficacy in DLBCL. Recent studies of B-cell leukemia have observed intron 2 retention associated with CAR T failure (Rabilloud T, et al. Nature communications. 2021 12(1):865), but this has not been examined in DLBCL there have been no reports of CAR T resistance associated with CD19 intron 6 retention in any context. CD19 intron 6 retention was one of the top hits associated with CAR T resistance in our cohort, confirmed on Integrated Genome Viewer (IGV) mapping of reads in patients detected as having high-vs. low-CD19 intron 6 retention (FIG. 5A). Given recent reports of intron 2 retention in BALL (leukemia), all 14 introns of CD19 were examined and percent intron retention compared between durable responders (DR) and non-durable responders (NDR; FIG. 5B), which showed significant retention of introns 2 and 6 associated with CAR T resistance (NDR). Of note intron 12 inclusion was seen in both DR and NDR and was not associated with CAR T response. Progression-free survival was significantly higher in patients within the lowest vs. highest tertile of intron 2 (top) or intron 6 (bottom) inclusion (FIG. 50; left). In addition to introns 2 and 6 being observed in the clinical cohort (FIG. 5C; DR and NDR), the presence of variable intron 2 and 6 retention was confirmed in lymphoma cell lines within CCLE. It was validated that introns 2 and 6 undergo partial retention within EJ-1 and OC-Ly3 cell lines using RT-PCR. Intron retention was less frequent within the de novo (untreated/low risk) TCGA cohort and higher within the NCI-CCR cohort. Of note, all of the shown datapoints/groups shown to have CD19 intron retention had NOT been exposed to CAR T therapy, indicating that CD19 intron retention occurs in DLBCL as part of its natural pathology even in the absence of CAR T-cell selection pressure.

### Summary

Using an institutional cohort, the first genome-wide investigation of aberrant mRNA splicing associated with CD19 CAR T therapy resistance in DLBCL was conducted. Several exon skipping and intron retention events were identified that were strongly associated with CAR T therapy response, involving several top genes with known roles in apoptosis, DNA-damage, oncogenic signaling, and immune response.

These findings, combined with clinical observations, have led to the hypothesis for the existence of 1) common resistance mechanisms that affect both radiation and CAR T therapy response (e.g., DNA damage, apoptosis, c-Myc) as well as 2) CAR T-specific mechanisms of resistance (e.g., CD19 intron retention and immune signaling), that may only impact CAR T resistance.

FBXW7 exon 2 skipping was chosen as an example of a common resistance mechanism, and the ability to induce exon 2 skipping was confirmed in several lymphoma cell lines using splice-switching ASOs and that exon 2 exclusion resulted in increased sensitivity to both radiation and CAR T-cell killing in vitro.

CD19 was selected for the CAR T only resistance example, and the data indicated that retention of introns 2 and 6 are both strongly associated with resistance and are expressed in several other cohorts.

### Example 3

### Methods

### Patient RNAseq Libraries—

Tumor biopsies were sampled from patients with relapsed and refractory diffuse large B-cell lymphoma. Clinical details for patients are provided in Supplementary Table 1. In total, 53 RNAseq libraries were analyzed from 48 patients receiving Yescarta. Of these libraries, 40 were generated from pre-treatment samples and 13 were generate from post-treatment samples. Of the 40 pre-treatment libraries, 3 were discarded for failing quality control thresholds. The remaining 37 pre-treatment libraries were used for analyses.

Samples were collected institutional review board approved protocols and research was done with adherence to the Declaration of Helsinki. Patients exhibiting a durable response (DR) were defined as those with continued remission 9 months after Yescarta infusion and patients that went into remission or died less than 9 months following Yescarta infusion were classified as exhibiting a non-durable response (NDR).

### RNAseq Quality Control and Alignment—

Adapters were removed from sequenced reads with cutadapt 1.16 (Martin, 2011) with parameters -m 30 -a AGATCGGAAGAGCACACGTCAGAACTCCAGTCAC-A AGATCGGAAGAGCGTCGTGTAGGGAAAGAGTGTA-trim-n (SEQ ID NO: 4 and 5, respectively). Then, reads were aligned to the Gencode v36 primary assembly of the human reference genome using the accompanying Gencode v36 primary assembly annotations using STAR (Dobin et al., 2013) version 2.5.3a with parameters—outFilterMismatchNoverLmax 0.04—chimSegmentMin 10—chimOutType SeparateSAMold-sjdbGTFfile gencode.v36.primary_assembly.annotation.gff3—twopassMode Basic. Aligned reads were filtered with Samtools (Li et al., 2009) version 1.15 with parameters -q 30 -F 256.

### Differential Alternative Splicing Analysis—

To identify differential splicing between pre-treatment samples from DR and NDR patients receiving Yescarta, filtered aligned reads were used as inputs for rMATS (Shen et al., 2014) turbo version 4.1.0 with parameters—gtf gencode.v36.primary_assembly.annotation.gtf—readLength 76—variable-read-length—novelSS—libType fr-secondstrand. The JCEC results files were then filtered requiring differential splicing events to have an FDR<0.05, ΔPSI >10%, and mean coverage of at least 10 reads for both forms of the splicing event. The PSI values for differential AS events in each sample were extracted from the filtered results files for additional analyses.

### Gene Expression Quantification—

To quantify expression at the gene level in the patient RNAseq libraries, the htseq-count module of HTSeq (Anders, Pyl, & Huber, 2015) version 0.11.2 was utilized to generate gene counts for each library with parameters -r pos -s yes -i gene_name<filtered alignments>gencode.v36.primary_assembly.annotation.gtf. The counts for each library were then consolidated into a single matrix. The counts were then normalized with DESeq2 and used for downstream analyses.

### TCGA-DLBCL RNAseq Data—

TCGA-DLBCL RNAseq libraries were acquired from the GDC Data Portal with controlled access granted under Project ID 6757. To quantify alternative splicing in this cohort, rMATS (Shen et al., 2014) was run on all samples with parameters—readLength 101—variable-read-length—novelSS—nthread 8—libType fr-unstranded—statoff—gtf gencode.v36.primary_assembly.annotation.gtf. Additionally, gene expression quantification was performed as above for the same samples.

### NCICCR-DLBCL RNAseq Data—

NCICCR-DLBCL RNAseq libraries were acquired from the GDC Data Portal (study accession phs001444; NIH dbGaP #23872). Using the accompanying metadata, we selected pre-treatment samples for analyses. Dropped ˜15 because of bad QC, need to include details. To quantify alternative splicing in this cohort, rMATS (Shen et al., 2014) was run on all samples with parameters—readLength 101—variable-read-length—novelSS—nthread 8—libType fr-unstranded—statoff—gtf gencode.v36.primary_assembly.annotation.gtf. Additionally, gene expression quantification was performed as above for the same samples.

### CCLE-DLBCL RNAseq Data—

CCLE-DLBCL RNAseq libraries were accessed from the GDC legacy archive. The downloaded libraries were aligned to a different reference genome than the one used in our experiments, therefore they were converted back to FASTQ format and aligned to the Gencode v36 primary assembly. The aligned reads were sorted by name and then converted back to FASTQ format with the bamToFastq utility within BEDTools (Quinlan & Hall, 2010). Then, reads were aligned to the Gencode v36 primary assembly of the human reference genome using the accompanying Gencode v36 primary assembly annotations using STAR (Dobin et al., 2013) version 2.5.3a with parameters—outFilterMismatch NoverLmax 0.04—chimSegmentMin 10—chimOutType SeparateSAMold-sjdbGTFfile gencode.v36.primary_assembly.annotation.gff3—twopassMode Basic. Aligned reads were filtered with Samtools (Li et al., 2009) version 1.15 with parameters -q 30-F 256.

### Secondary Intron Retention Calculations—

Intron retention events in CD19 were quantified by a secondary, more robust method in patient samples and the NCICCR-DLBCL cohort using the splice junction coverage and per-base median read coverage of introns. CD19 intron 6 overlaps exon 1 of RABEP2 and any reads potential mapping to RABEP2 were removed. Then filtered alignments were used as inputs into Samtools (Li et al., 2009) depth to obtain base-level read coverage of the intron loci. Splice junction coverage was quantified from the filtered alignments using the sjFromSAMcollapseUandM.awk in the STAR (Dobin et al., 2013) package. Intron retention levels were calculated by dividing the median intron coverage by the splice junction reads plus the median intron coverage. The pipeline of the scripts used in the method can be found at github.com/jeraldnoble/CAR-T_scripts.

### Normal CD19 Transcript Abundance—

We estimated the proportion of CD19 transcripts that do not retain introns 2 and 6 via:

$$\frac{( 1 - {Ψ}_{Intron 2} ) + ( 1 - {Ψ}_{Intron 6} )}{2}$$

where:
Ψ_{Intron2} is the PSI value of CD19 intron 2.
Ψ_{Intron6} is the PSI value of CD19 intron 6.

### Statistical Analyses

All statistical analyses were conducted in R version 4.2.1. Logistical regression models were constructed using the glm function with the argument “family=“binomial””. Survival analyses were done using the survival and survminer libraries. An R markdown notebook containing the code used for all statistical analyses and relevant figures can be found in Supplementary File.

### Results

### Retention of CD19 Intron 6 is Significantly Upregulated in NDR Patients—

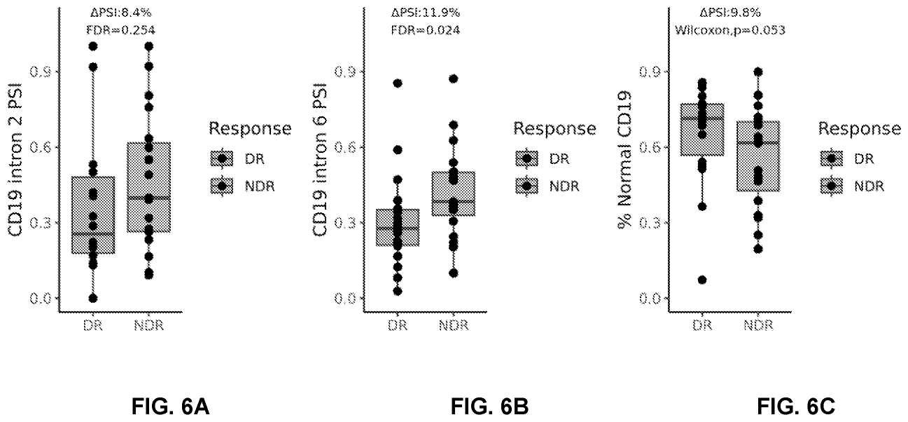

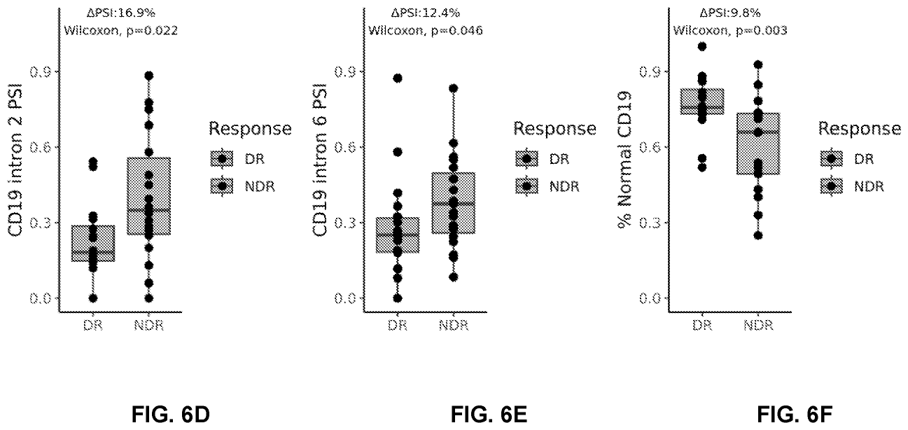

Initially we assessed if CD19 was differentially expressed between DR and NDR patients but found the gene-level expression differences not statistically significant (FIG. 6A). the skipping of CD19 exon 2 (Δex2) between DR and NDR patients was investigated showing that it was not differentially spliced (Sotillo et al., 2015). Upregulated retention of intron 2 has been characterized as associated with poor response in pediatric B-cell acute lymphoblastic leukemia (B-ALL) patients receiving CAR-T therapy and the decreased CD19 surface expression in Raji cell lines (Asnani et al., 2020). Intron 2 was more highly retained in NDR samples (FIG. 6C), but not statistically significant. The differential splicing results from rMATS (Shen et al., 2014) between DR and NDR patients detected a significant increased retention of CD19 intron 6 in NDR patients with FDR 0.02 and ΔPSI 11.9%. Using the PSI values generated by rMATS (Shen et al., 2014) the percentage of CD19 transcripts not expressing introns 2 or 6 was calculated. The percentage of normal CD19 transcripts was different between patients (ΔPSI 11.9%) but was not statistically significant using rMATS PSI values (FIG. 6C).

To calculate intron retention levels in CD19 more thoroughly, we developed a pipeline to quantify PSI using median base-level coverage of the introns. Using this methodology, both introns 2 and 6 were significantly upregulated (FIG. 6D-6E, respectively) in NDR patients. Additionally, the percentage of normal transcripts calculated using PSI values generated from this method were significantly upregulated in DR patients (FIG. 6F).

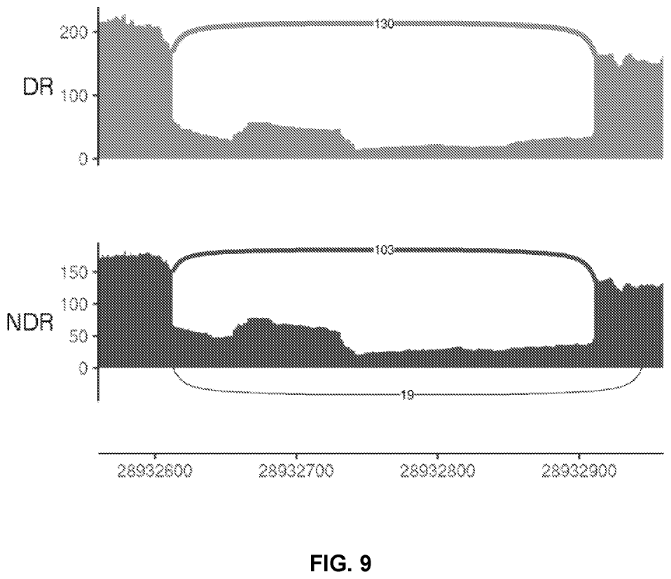

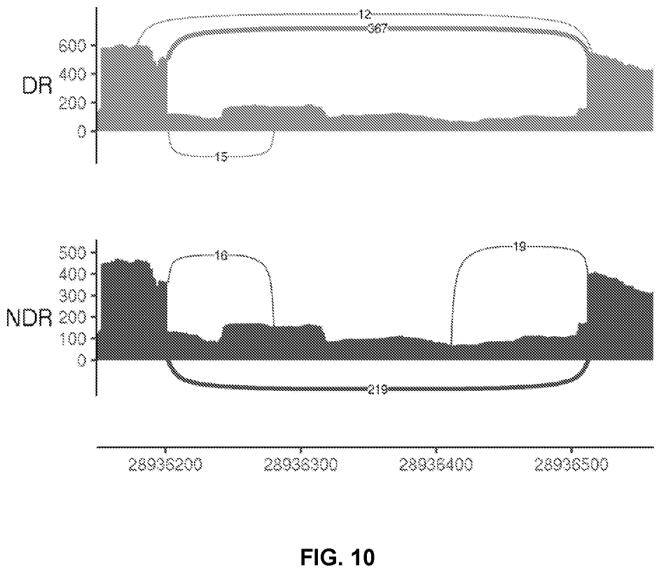

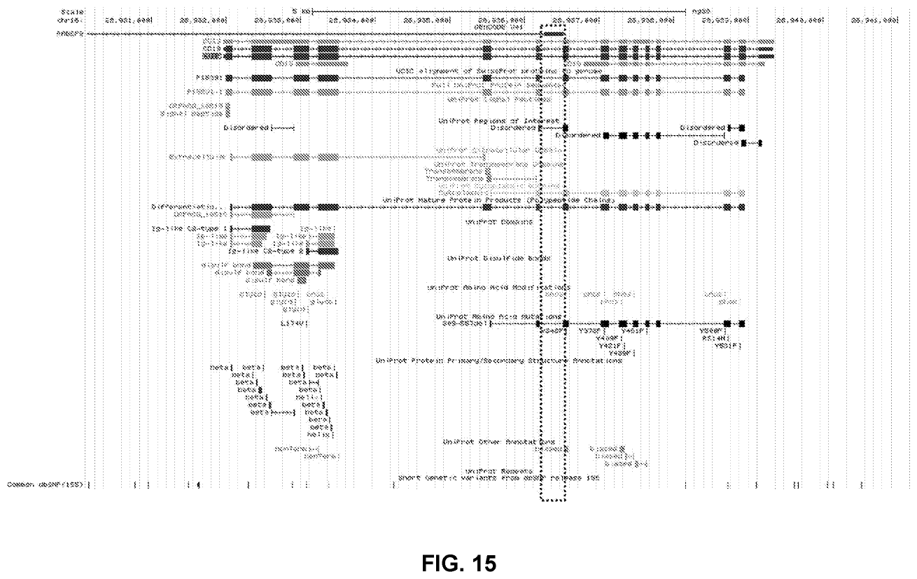

The amino acid sequence of intron 6 in CD19 contains several stop codons which may result in a protein with a truncated cytoplasmic domain (FIG. 15) or an mRNA isoform that will be targeted by the nonsense mediated decay pathway (NMD). Per-base coverage of intron 6 was similar between DR and NDR patients, but DR patients expressed 79% (FIG. 9) more junction spanning reads supporting the splicing out of intron 6 and therefore potentially functional CD19 isoforms. Per-base coverage of CD19 intron 2 in DR and NDR patients is depicted in FIG. 10.

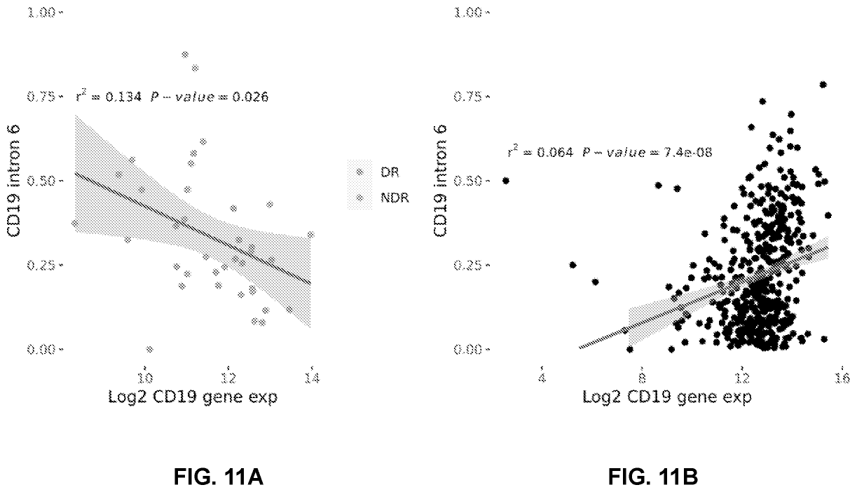

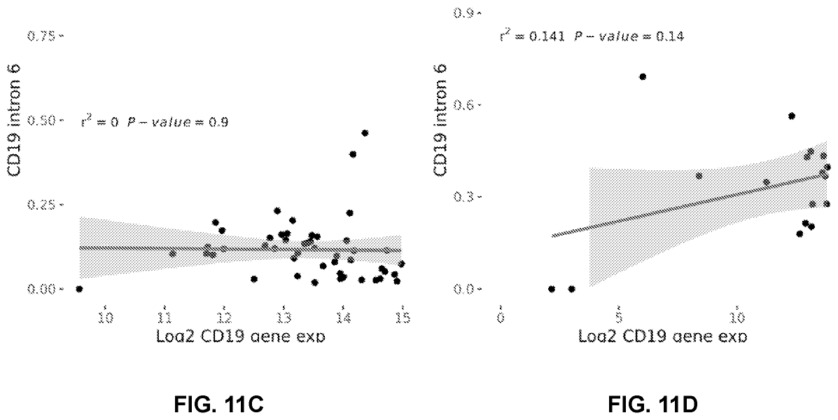

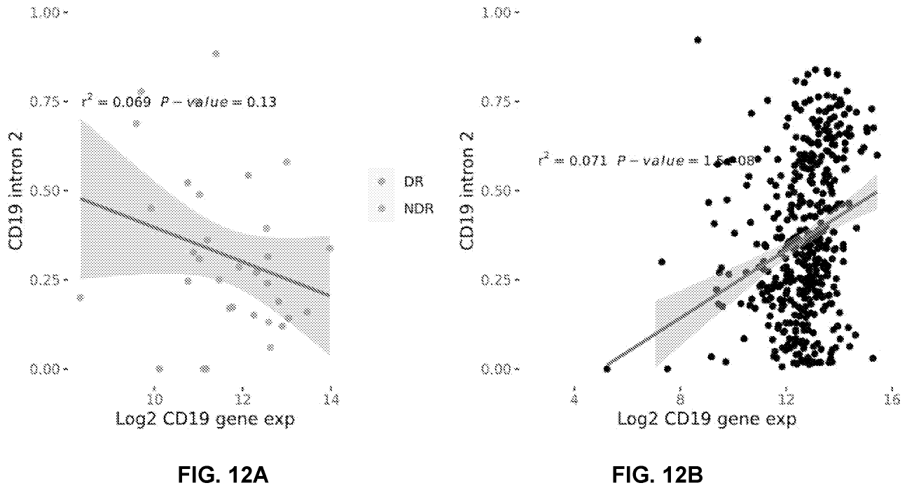

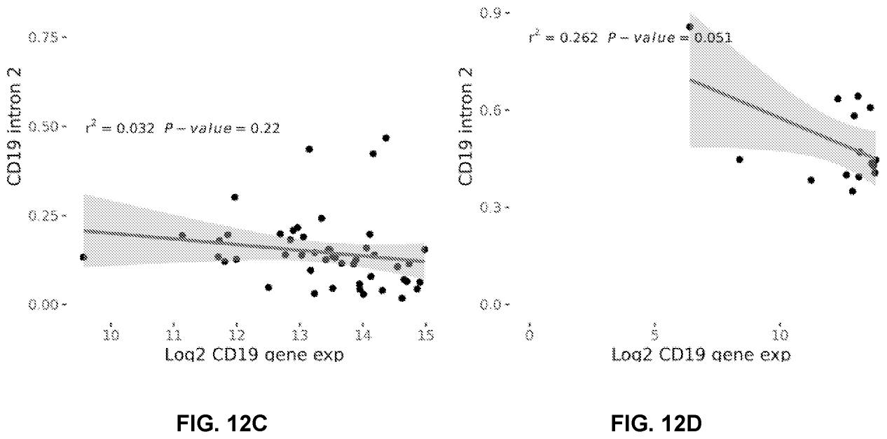

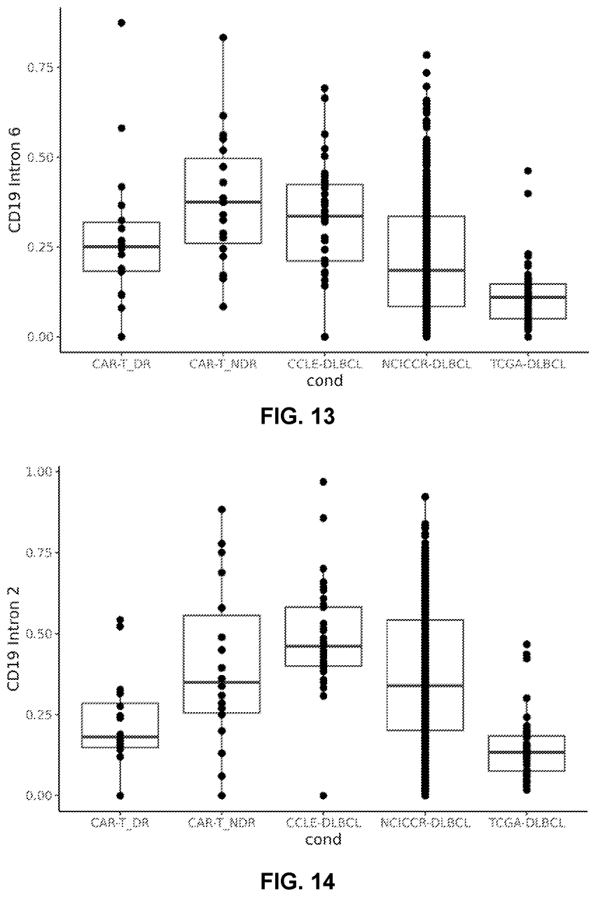

The expression of CD19 at the gene level showed a slightly negative relation (r2=0.134) with CD19 intron 6 retention in the CAR-T patient cohort (FIG. 11A). This same trend was not observed in the NCICCR-DLBCL, TCGA-DLBCL, and CCLE-DLBCL datasets (FIG. 11B-11D). The relation between CD19 gene expression and CD19 intron 2 retention followed a similar trend in these datasets (FIG. 12). The PSI values of CD19 introns 6 and 2 in the same datasets are summarized in FIGS. 13 and 14 respectively.

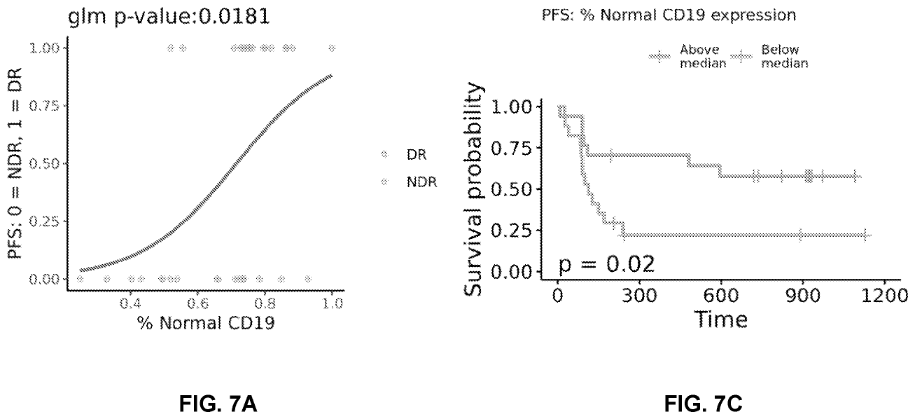

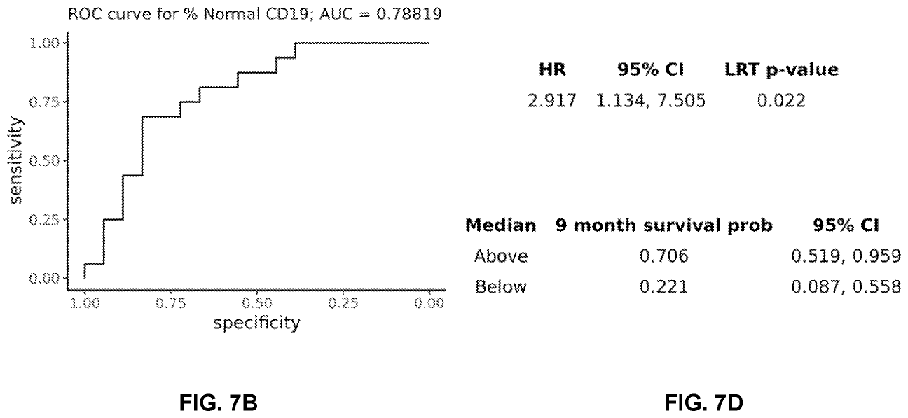

To assess how well the percentage of normal CD19 transcripts classify durable response, a logistic regression model was constructed predicting durable response using the percentage of normal CD19 transcripts and yielded a p-value 0.0181. The performance of this model to predict durable response using percentage of normal CD19 transcripts is conveyed in FIG. 7A. Leave one out cross validation was performed on this model yielding an overall accuracy of 67.6%. Sensitivity (predicting NDR) was 61.1% and specificity (predicting DR) was 81.3%. The ROC curve summarizing these findings is displayed in FIG. 7B.

Normal CD19 Transcript Expression is Associated with Progress Free Survival—

To assess the impact of CD19 intron retention on survival in patients following Yescarta treatment, patients were separated into groups expressing normal CD19 transcript levels above and below the median expression level for all samples. Expression of normal CD19 transcripts above the median level was significantly associated with progress free survival (PFS) (FIG. 7C). Patients expressing normal CD19 transcripts above the median level had a 70.6% (95% CI: 0.52-0.96) probability of DR while patients expressing these transcripts below the median level had a 22.1% (95% CI: 0.09-0.56) probability of NDR (FIG. 7D).

### RNA Binding Proteins Associated with Retention of CD19 intron 6

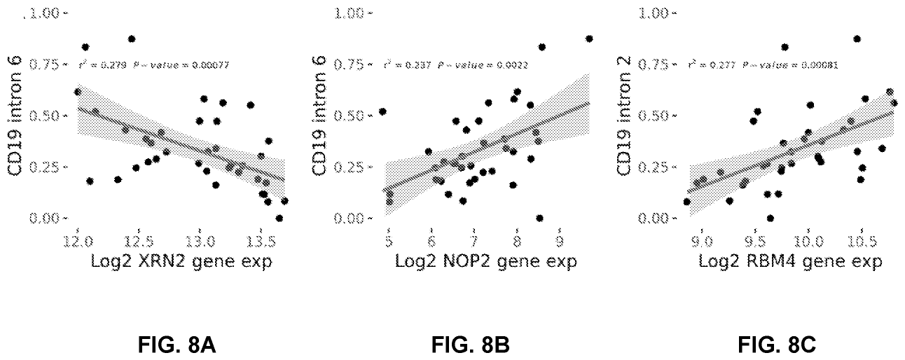

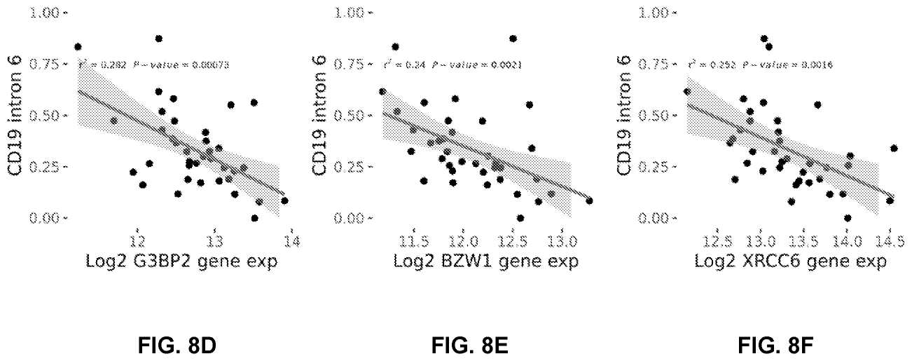

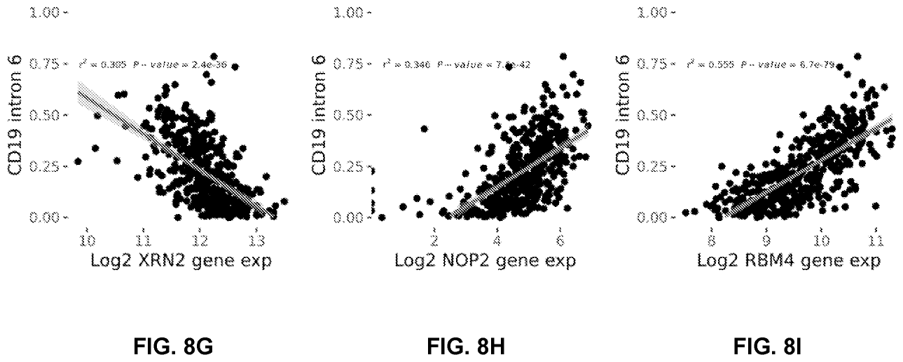

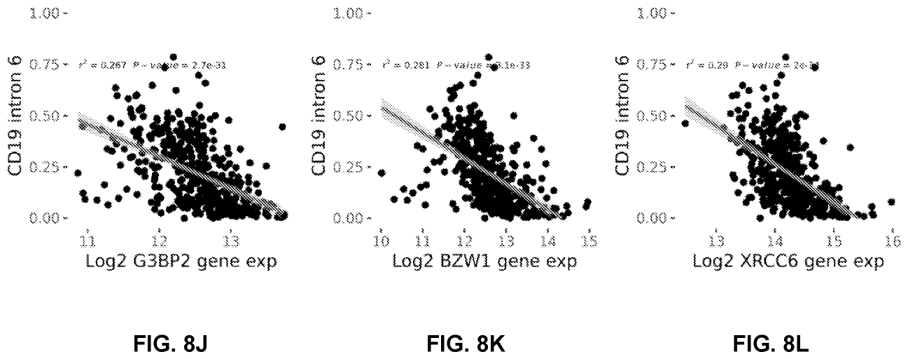

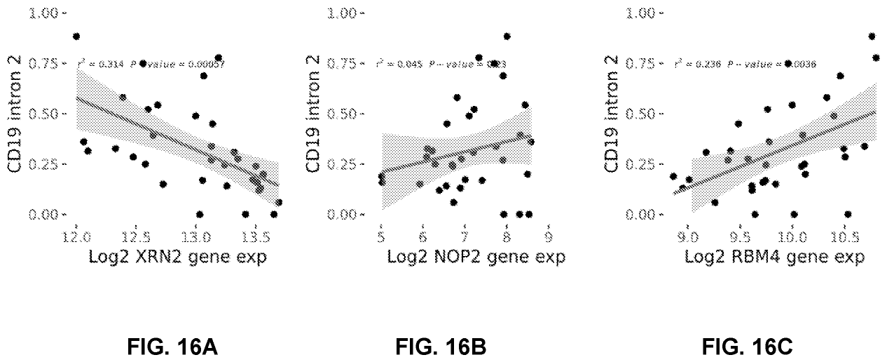

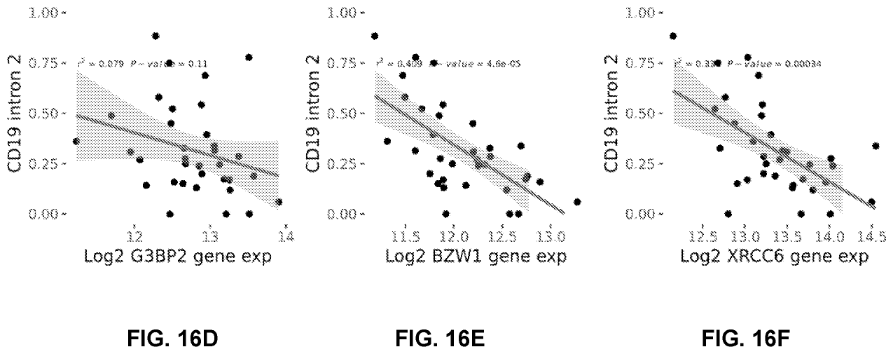

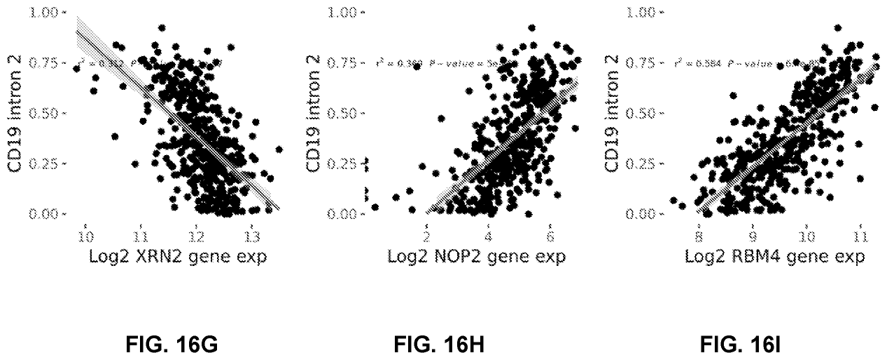

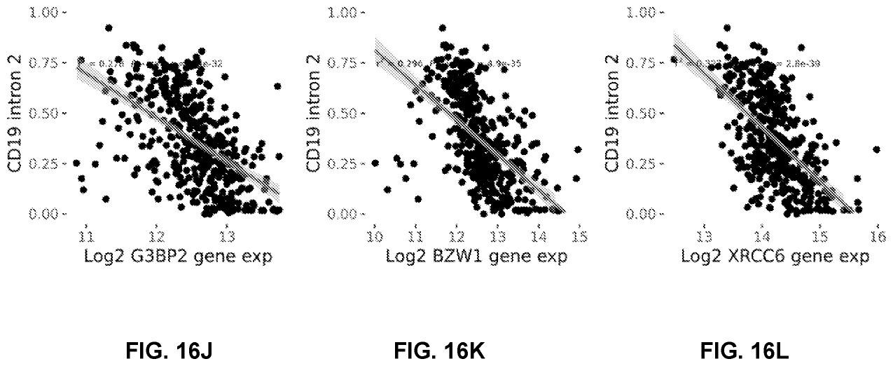

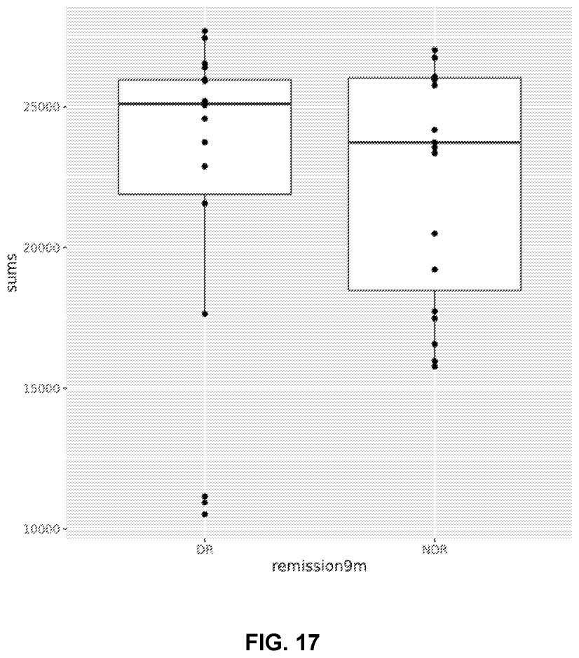

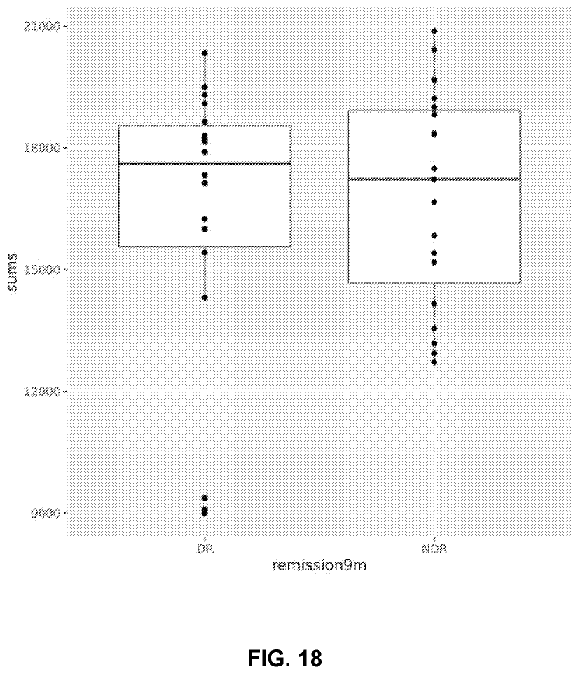

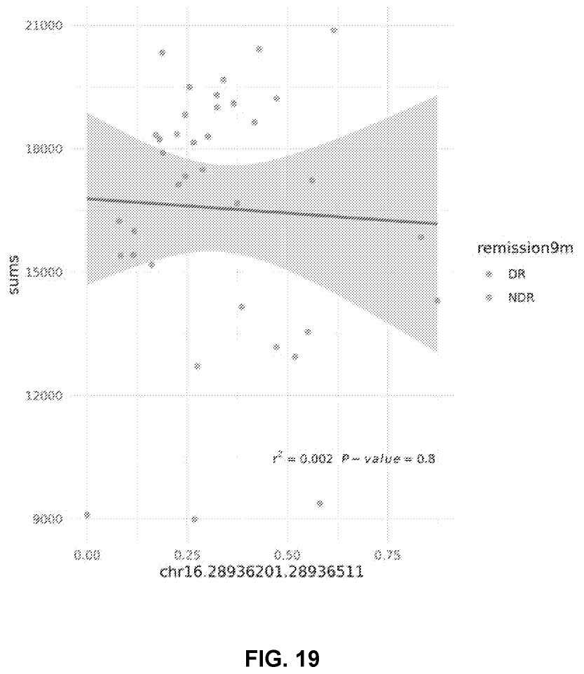

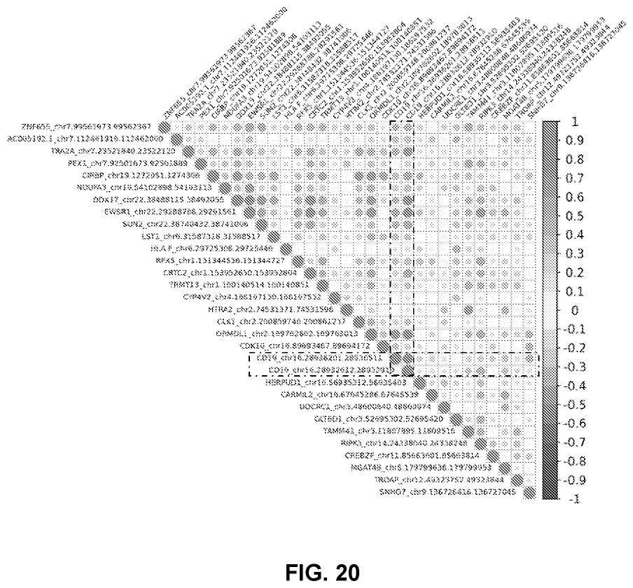

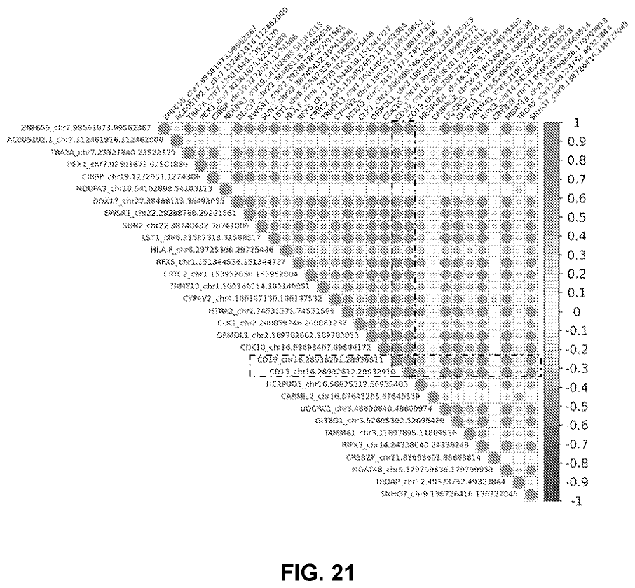

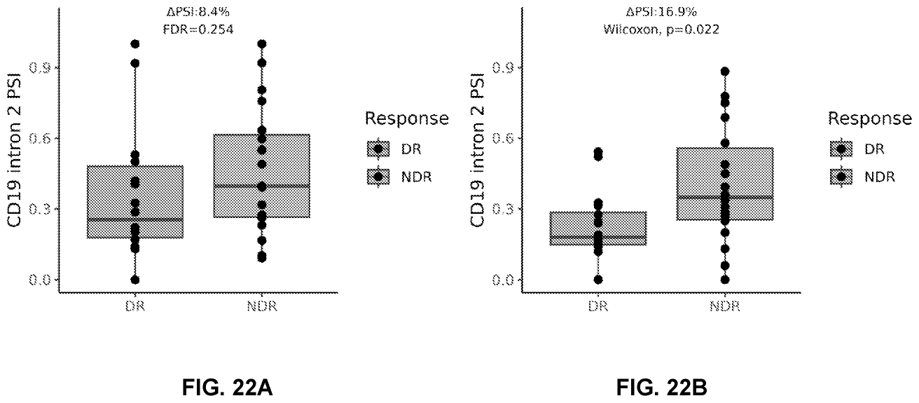

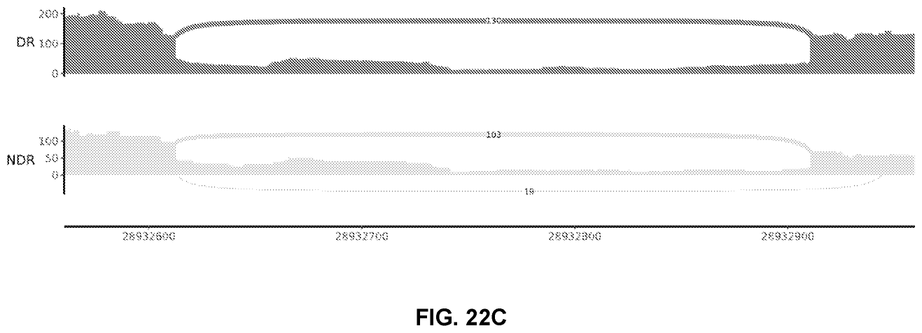

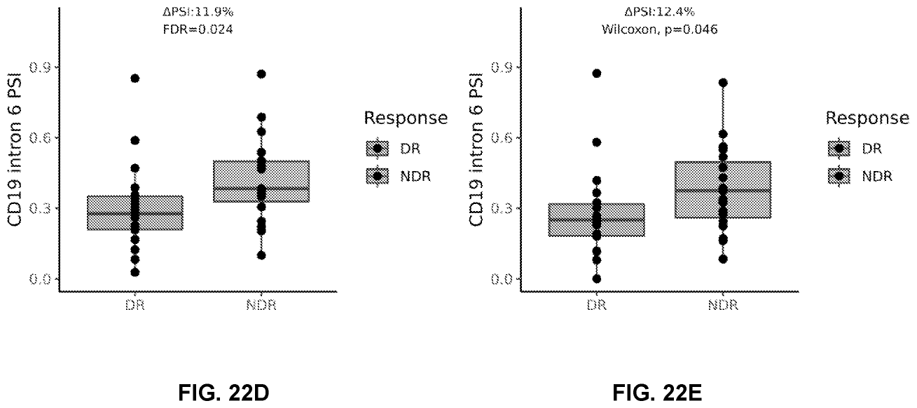

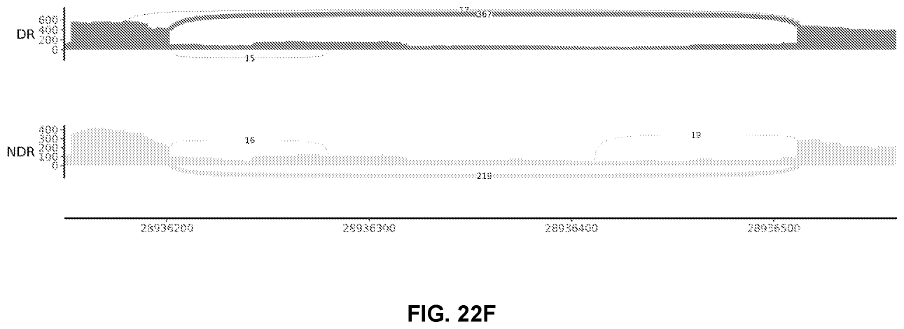

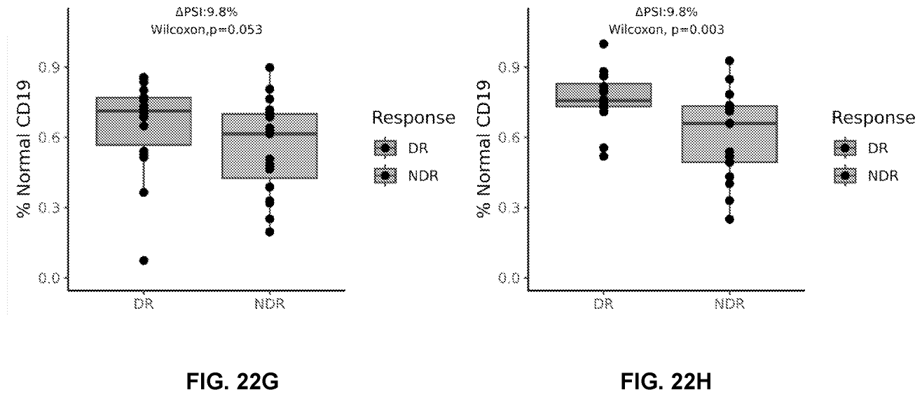

To identify putative upstream regulators of CD19 intron 6 retention, we ran a simple linear regression in R measuring the relationship between the gene expression (Methods) of 499 RNA binding proteins and CD19 intron 6 PSI. Additionally, we ran the same analysis in the NCICCR-DLBCL cohort to confirm the findings in a substantially larger cohort. 6 RNA binding proteins were identified, XRN2, NOP2, RBM4, G3BP2, BZW1, and XRCC6 with a significant (r²>0.2 and p-value <0.05) relation between their gene expression and CD19 intron 6 retention in both the CAR-T and NCICCR-DLBCL cohorts (FIG. 8). The relation between the expression of these genes and CD19 intron 2 is summarized in FIG. 16.

### Discussion

Alternative splicing of CD19 has been associated with failure in multiple CD19-directed therapies. The skipping of exon 2 results in the expression of a CD19 isoform with an N-truncated extracellular domain that is not recognized by CD19 flow cytometry antibodies and is substantially less targeted to the cell membrane (Sotillo et al., 2015). The intraexonic skipping of exon 2 causes a frame shift deletion that results in a premature stop codon within exon 2 and was more skipped in pre-treatment B-ALL patients receiving blinatumomab that did not respond to treatment (Zhao et al., 2021). The retention of intron 2 in CD19 introduces a premature stop codon into the mRNA transcript and results in lower cell surface protein expression of CD19 (Asnani et al., 2020). However, the analyses of intron 2 was conducted in two patients from the Sotillo et al. (2015) cohort. Here the splicing of intron 2 and 6 in a pre-CAR-T treatment R/R DLBCL cohort of 37 patients is presented. While intron 2 retention was upregulated in NDR patients, it was not statistically significant. Intron 6 was identified as being significantly upregulated in NDR patients and posit that the downstream effect is similar to the retention of intron 2 because they both harbor stop codons. The retention of intron 6 is thus a novel mechanism of failure for R/R DLBCL patients receiving CD19-directed CAR-T therapy.

Genetic mutations and loss of heterozygosity were associated with CD19 negative relapse in post-treatment B-ALL samples receiving Tisagenlecleucel, but aberrant splicing in CD19 was not detected (Orlando et al., 2018). In this study mutations were found in exons 2-5 spanning the extracellular domain and part of the transmembrane domain. Although intron 6 exists within the cytosolic domain, and should allow the extracellular and transmembrane domains to remain functional, it is associated with the decreased surface expression of CD19.

RNA binding proteins function as upstream AS regulatory factors and perturbation in their expression can affect gene expression and alternative splicing (Van Nostrand et al., 2020). More specifically, knockdown of SRSF3 increases skipping of CD19 exon 2 (Sotillo et al., 2015) and knockdown of PTBP1 increases the retention of CD19 intron 2 (Cortes-Lopez et al., 2022)

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for preventing or reversing CAR-T cell resistance and/or radioresistance in a relapsed and refractory diffuse large B-cell lymphoma (R/R DLBCL) of a subject, comprising
assaying a sample from the subject for mRNA sequences of genes with roles in DNA damage, apoptosis, immune activation, and/or c-MYC signaling;
detecting aberrant splicing in one or more of the mRNA sequences; and
administering to the subject an antisense oligonucleotide (ASO) that prevents the aberrant splicing.

2. The method of claim 1, wherein the gene comprises an FBXW7 gene, and wherein the aberrant splicing comprises exon 2 retention, and wherein the ASO promotes exon 2 skipping.

3. The method of claim 2, wherein the ASO comprises the nucleic acid sequence
(SEQ ID NO: 1)
GGCCACTCACACTTTTAGAAAAGAG.

4. The method of claim 1, wherein the gene comprises CD19, and wherein the aberrant splicing comprises intron 2 retention, and wherein the ASO promotes intron 2 skipping.

5. The method of claim 4, wherein the ASO comprises the nucleic acid sequence
(SEQ ID NO: 2)
AACAGCTCCCCTGGGAAGAGACCCA.

6. The method of claim 1, wherein the gene comprises CD19, and wherein the aberrant splicing comprises intron 6 retention, and wherein the ASO promotes intron 6 skipping.

7. The method of claim 1, wherein the gene comprises ATG16L1, and wherein the aberrant splicing comprises intron 13 skipping, and wherein the ASO promotes intron 13 retention.

8. The method of claim 6, wherein the ASO comprises the nucleic acid sequence G ACTGAATTTCCTCACAGACTTTGC (SEQ ID NO:3).


SEQUENCE LISTING (5 sequences)

SEQ ID NO: 1 - DNA, 25 bp, synthetic construct
  source 1..25: mol_type = other DNA
ggccactcacacttttagaaaagag

SEQ ID NO: 2 - DNA, 25 bp, synthetic construct
  source 1..25: mol_type = other DNA
aacagctcccctgggaagagaccca

SEQ ID NO: 3 - DNA, 24 bp, synthetic construct
  source 1..24: mol_type = other DNA
actgaatttcctcacagactttgc

SEQ ID NO: 4 - DNA, 34 bp, synthetic construct
  source 1..34: mol_type = other DNA
agatcggaagagcacacgtcagaactccagtcac

SEQ ID NO: 5 - DNA, 34 bp, synthetic construct
  source 1..34: mol_type = other DNA
agatcggaagagcgtcgtgtagggaaagagtgta